(19) United States
(12) Patent Application Publication
(10) Pub. No.: US 2024/0024261 A1
(43) Pub. Date: Jan. 25, 2024

(54) PHARMACEUTICAL COMPOSITION FOR IMPROVING COGNITIVE ABILITY OR ENHANCING MEMORY

(71) Applicant: DR. RAYMOND LAB INC, Yuseong-gu, Daejeon (KR)

(72) Inventor: Byeong Deog PARK, Yuseong-Gu, Daejeon (KR)

(21) Appl. No.: 18/024,967

(22) PCT Filed: Sep. 17, 2021
(86) PCT No.: PCT/IB2021/058477

§ 371 Date: Mar. 6, 2023

(30) Foreign Application Priority Data
Sep. 21, 2020 (KR) 10-2020-0121453
Sep. 16, 2021 (KR) 10-2021-0123961

(51) Int. Cl. A61K 31/16; A61K 9/00; A61P 25/28; A61K 31/445; A61K 47/14

(52) U.S. Cl. CPC A61K 31/16; A61K 9/0053; A61P 25/28; A61K 31/445; A61K 47/14

(57) ABSTRACT

The present invention relates to a pharmaceutical composition for improving cognitive ability or enhancing memory, and more specifically, to a health functional food for improving cognitive ability or enhancing memory, and a composite pharmaceutical composition for improving cognitive ability or enhancing memory, which are mixed compositions, respectively, for co-administration with other active materials exhibiting an effect of improving cognitive ability or enhancing memory. The pharmaceutical composition for improving cognitive ability or enhancing memory, according to the present invention, has been confirmed to exhibit an effect of significantly reducing cognitive impairment or memory loss caused by neurodegenerative brain diseases or inflammatory brain diseases such as autism, Alzheimer's disease, Parkinson's disease, peripheral neuropathy, amyotrophic lateral sclerosis, ischaemia, stroke and multiple sclerosis, etc.

### TECHNICAL FIELD

The present invention relates to a pharmaceutical composition for improving cognitive ability or enhancing memory, and more particularly, to a mixed composition for being used in combination with other active materials having an effect of improving cognitive ability or enhancing memory, which is a health functional food for improving cognitive ability or enhancing memory or a composite pharmaceutical composition for improving cognitive ability or enhancing memory.

### BACKGROUND ART

Ceramide metabolism is related to various neurodegenerative brain diseases. As an example, a result has been reported showing that in autistic patients, a total amount of ceramide is greatly decreased in the brain and skin (Shin et al., Phenotypic overlap between atopic dermatitis and autism, BMC Neuroscience 22:43, 2021).

In addition, in various neurodegenerative brain diseases, metabolic abnormalities of ceramide and sphingolipids appear, and since it is known that sphingosine-1-phosphate plays an important role at this time, research on a therapeutic agent for Alzheimer's disease is in progress (Alessenko and Albi, Exploring sphingolipid implications in neurodegeneration, Frontiers in Neurology 11:437, 2020).

As an agonist of a cannabinoid receptor CB1 involved in ceramide metabolism, research results have been reported showing that an N-palmitoyl ethanolamide (PEA) and N-palmitoyl serinol promote synthesis of ceramide and increase the activity of serine palmitoyltransferase (SPT) in a sphingosine synthesis step (Shin et al., N-palmitoyl serinol stimulates ceramide production through a CB1-dependent mechanism in in vitro model of skin inflammation, Int. J. Mol. Sci 22, 8302, 2021).

It has been reported that when PEA acting as the agonist of CB1 is orally administered to an autistic patient, an effect is shown (Nicola Antonucci, et al Beneficial Effects of Palmitoylethanolamide on Expressive Language, Cognition, and Behaviors in Autism: A Report of Two Cases, Hindawi Publishing Corporation Case Reports in Psychiatry Volume 2015, Article ID 325061, 6 pages), and it has been revealed that N-palmitoyl serinol (trade name: Neuromide) also acts as the agonist of CB1 and has an effect limited to skin application such as atopy (Park, Byung-deok, etc., Korean Patent Registration No. 10-1983566), (Si Wen, Li Ye, Dan Liu, Bin Yang, Mao-Qiang Man, Topical N-palmitoyl serinol, a commensal bacterial metabolite, prevents the development of epidermal permeability barrier dysfunction in a murine model of atopic dermatitis-like skin, The Canadian Journal of Veterinary Research 201, 2021; 85:201-204).

The present researchers studied applicability of N-palmitoyl serinol which is a CB1 agonist (trade name: Neuromide®) to neurodegenerative brain diseases, and confirmed that when Neuromide was orally administered, ceramide synthesis was promoted and the amount of sphingosine-1-phosphate which was significantly decreased in neurodegenerative brain disease patients was increased, and also confirmed decreased activity of acetylcholinesterase which is an acetylcholine decomposition enzyme, an increased amount of acetylcholine, and a decrease in TNF-α and Il-1β which are inflammatory cytokines in various indicators of brain function, and thus, newly found that N-palmitoyl has an effect of improving or treating neurodegenerative brain diseases, thereby completing the present invention.

### RELATED ART DOCUMENT

(Patent Document 0001) Korean Patent Registration Publication No. 1983566

(Non-Patent Document 0001) Cohen L. et al. Nature 549:48-53 (2017).

(Non-Patent Document 0002) Holscher C. CNS Drugs 26(10):871-882 (2012).

(Non-Patent Document 0003) Shin et al., Phenotypic overlap between atopic dermatitis and autism, BMC Neuroscience 22:43, 2021

(Non-Patent Document 0004) Alessenko and Albi, Exploring sphingolipid implications in neurodegeneration, Frontiers in Neurology 11:437, 2020

(Non-Patent Document 0005) Shin et al., N-palmitoyl serinol stimulates ceramide production through a CB1-dependent mechanism in in vitro model of skin inflammation, Int. J. Mol. Sci 22, 8302, 2021.

(Non-Patent Document 0006) Nicola Antonucci, et al Beneficial Effects of Palmitoylethanolamide on

Expressive Language, Cognition, and Behaviors in Autism: A Report of Two Cases, Hindawi Publishing Corporation Case Reports in Psychiatry Volume 2015, Article ID 325061, 6 pages

(Non-Patent Document 0007) Si Wen, Li Ye, Dan Liu, Bin Yang, Mao-Qiang Man, Topical N-palmitoyl serinol, a commensal bacterial metabolite, prevents the development of epidermal permeability barrier dysfunction in a murine model of atopic dermatitis-like skin, The Canadian Journal of Veterinary Research 201, 2021; 85:201-204.

### DISCLOSURE

### Technical Problem

An object of the present invention is to provide a pharmaceutical composition for improving cognitive ability or enhancing memory including N-palmitoyl serinol as an effective component.

Another object of the present invention is to provide a health functional food for improving cognitive ability or enhancing memory including N-palmitoyl serinol as an effective component.

Another object of the present invention is to provide a composite pharmaceutical composition for improving cognitive ability or enhancing memory including N-palmitoyl serinol and an active material for improving cognitive ability or enhancing memory.

Another object of the present invention is to provide a method of preparing the composite pharmaceutical composition for improving cognitive ability or enhancing memory.

Still another object of the present invention is to provide a method of administering a pharmaceutical composition for improving cognitive ability or memory decline.

### Technical Solution

The pharmaceutical composition for improving cognitive ability or enhancing memory according to the present invention includes N-palmitoyl serinol as an effective component.

In the pharmaceutical composition for improving cognitive ability or enhancing memory according to an exemplary embodiment of the present invention, the pharmaceutical composition may be for improving or treating neurodegenerative brain diseases or neuroinflammatory diseases.

In the pharmaceutical composition for improving cognitive ability or enhancing memory according to an exemplary embodiment of the present invention, the disease may be selected from the group consisting of autism, Alzheimer's disease, Parkinson's disease, peripheral neuropathy, amyotrophic lateral sclerosis, ischaemia, stroke, and multiple sclerosis.

In the pharmaceutical composition for improving cognitive ability or enhancing memory according to an exemplary embodiment of the present invention, the pharmaceutical composition may be a formulation of a preparation for oral administration or a preparation for parenteral administration.

In the pharmaceutical composition for improving cognitive ability or enhancing memory according to an exemplary embodiment of the present invention, the formulation of a preparation for oral administration may be tablets, pills, hard/soft capsule, liquid, suspension, emulsifier, syrup, granules, or elixirs.

In the pharmaceutical composition for improving cognitive ability or enhancing memory according to an exemplary embodiment of the present invention, the formulation of a preparation for parenteral administration may be a sterile aqueous or oily solvent for intravenous, subcutaneous, sublingual, or intramuscular administration.

In another general aspect, a health functional food for improving cognitive ability or enhancing memory includes N-palmitoyl serinol as an effective component.

The health functional food for improving cognitive ability or enhancing memory according to an exemplary embodiment of the present invention may be powder, granules, tablets, capsules, or beverages.

In another general aspect, a composite pharmaceutical composition for improving cognitive ability or enhancing memory includes N-palmitoyl serinol and an active material for improving cognitive ability or enhancing memory.

In the composite pharmaceutical composition for improving cognitive ability or enhancing memory according to an exemplary embodiment of the present invention, the composite pharmaceutical composition may include first particles including N-palmitoyl serinol; and second particles including an active material for improving cognitive ability or enhancing memory.

In the composite pharmaceutical composition for improving cognitive ability or enhancing memory according to an exemplary embodiment of the present invention, the composite pharmaceutical composition may further include a sterol-based compound, a higher fatty acid, and a higher fatty acid alcohol.

In the pharmaceutical composition for improving cognitive ability or enhancing memory according to an exemplary embodiment of the present invention, the composition may be a mixture including 10 to 1000 parts by weight of the sterol-based compound, 100 to 3000 parts by weight of the higher fatty acid, 100 to 5000 parts by weight of the higher fatty acid alcohol, and 1 to 500 parts by weight of the active material with respect to 100 parts by weight of N-palmitoyl serinol.

In the pharmaceutical composition for improving cognitive ability or enhancing memory according to an exemplary embodiment of the present invention, the composite pharmaceutical composition may form a stable crystal lamella micro-encapsulation system (MES) or orthorhombic crystalline phase.

In the pharmaceutical composition for improving cognitive ability or enhancing memory according to an exemplary embodiment of the present invention, the active material may be any one or more selected from the group consisting of Donepezil, Rivastigmine, and Galantamine.

In another general aspect, a method of preparing a composite pharmaceutical composition for improving cognitive ability or enhancing memory includes: preparing first particles including N-palmitoyl serinol; and preparing second particles including an active material for improving cognitive ability or enhancing memory.

In the method of preparing a composite pharmaceutical composition for improving cognitive ability or enhancing memory according to an exemplary embodiment of the present invention, the active material may be included in the form of an active material or a pharmaceutically acceptable salt thereof.

In the method of preparing a composite pharmaceutical composition for improving cognitive ability or enhancing memory according to an exemplary embodiment of the present invention, the first particles and the second particles may be mixed at a weight ratio of 100:0.5 to 1.5.

In the method of preparing a composite pharmaceutical composition for improving cognitive ability or enhancing memory according to an exemplary embodiment of the present invention, the first particles and the second particles may release N-palmitoyl serinol and the active material continuously for 12 hours to 24 hours to maintain an effect of improving diseases related to cognitive impairment or memory loss.

In the method of preparing a composite pharmaceutical composition for improving cognitive ability or enhancing memory according to an exemplary embodiment of the present invention, the disease related to cognitive impairment or memory loss may be selected from the group consisting of autism, Alzheimer's disease, Parkinson's disease, peripheral neuropathy, amyotrophic lateral sclerosis, ischaemia, stroke, and multiple sclerosis.

In the method of preparing a composite pharmaceutical composition for improving cognitive ability or enhancing memory according to an exemplary embodiment of the present invention, the first particles and the second particles may be mixed in a uniform form to form a stable crystal lamella micro-encapsulation system (MES) or orthorhombic crystalline phase.

In still another general aspect, an administration method for improving cognitive ability or memory decline includes: administrating a pharmaceutical composition for improving cognitive ability or enhancing memory including N-palmitoyl serinol as an effective component; or a composite pharmaceutical composition for improving cognitive ability or enhancing memory including N-palmitoyl serinol and an active material for improving cognitive ability or enhancing memory to a subject.

In the administration method for improving cognitive ability or memory decline according to an exemplary embodiment of the present invention, a dose of the pharmaceutical composition for improving cognitive ability or enhancing memory or the composite pharmaceutical composition for improving cognitive ability or enhancing memory to be administered to the subject may be 1 to 200 mg/kg based on the weight of the subject.

In the administration method for improving cognitive ability or memory decline according to an exemplary embodiment of the present invention, an administration interval of the pharmaceutical composition for improving cognitive ability or enhancing memory or the composite pharmaceutical composition for improving cognitive ability or enhancing memory to be administered to the subject may be 12 hours or more and 240 hours or less.

In the administration method for improving cognitive ability or memory decline according to an exemplary embodiment of the present invention, the administration method may include oral administration, subcutaneous injection, and subcutaneous application.

### Advantageous Effects

The pharmaceutical composition for improving cognitive ability or enhancing memory according to the present invention has an effect of significantly improving cognitive impairment or memory loss caused by a neurodegenerative brain disease or an inflammatory brain disease such as autism, Alzheimer's disease, Parkinson's disease, peripheral neuropathy, amyotrophic lateral sclerosis, ischaemia, stroke, and multiple sclerosis, and is manufactured into a health functional food having various forms of formulation, thereby obtaining an effect of easy accessibility and intake.

In addition, it was confirmed that for the composite pharmaceutical composition for improving cognitive ability or enhancing memory according to the present invention, when one or more other active materials having an effect of improving cognitive ability or enhancing memory and N-palmitoyl serinol (trade name: Neuromide®) which is the effective component of the pharmaceutical composition for improving cognitive ability or enhancing memory of the present invention are mixed in an excipient form and treated in combination, the composite pharmaceutical composition has an effect of significantly improving cognitive ability and enhancing memory.

### DESCRIPTION OF DRAWINGS

FIG. 1 shows results of measuring latency time spent in a passive avoidance test as average±standard error.

Independent T-test for significant difference from a negative control group: *p<0.05, **p<0.01.

FIG. 2 shows results of measuring an acetylcholine content in a hippocampus as average±standard error.

Independent T-test for significant difference from a negative control group: **p<0.01.

FIG. 3 shows results of measuring activity of acetylcholine esterase (AchE) in a forebrain as average±standard error.

Independent T-test for significant difference from a negative control group: *p<0.05, **p<0.01.

FIG. 4 shows results of measuring TNF-α in a hippocampus as average±standard error.

Independent T-test for significant difference from a negative control group: *p<0.05, **p<0.01.

FIG. 5 shows results of measuring IL-1β in a hippocampus as average±standard error.

Independent T-test for significant difference from a negative control group: *p<0.05, **p<0.01.

FIG. 6 shows results of measuring an individual body weight during the test as average±standard error.

FIG. 7 shows distributions of ceramide by experimental groups G1, G2, and G5 and N-acyl chain length.

FIG. 8 shows distributions of the total weight of ceramide of experimental groups G1, G2, and G5.

FIG. 9 shows distributions of sphingoid base of experimental groups G1, G2, and G5.

### BEST MODE

Hereinafter, the composition for improving cognitive ability of the present invention will be described in detail with reference to the attached tables and drawings.

When a drawing is illustrated, the drawing is provided by way of example so that the idea of the present invention may be sufficiently conveyed to a person skilled in the art. Therefore, the present invention is not limited to the provided drawings, but may be embodied in many different forms, and the drawings may be exaggerated in order to clarify the spirit of the present invention.

Technical terms and scientific terms used herein have the general meaning understood by those skilled in the art to which the present invention pertains unless otherwise defined, and a description for the known function and configuration which may unnecessarily obscure the gist of the present invention will be omitted in the following description and the accompanying drawings.

In addition, the singular form used in the specification of the present invention may be intended to also include a plural form, unless otherwise indicated in the context.

In addition, units used in the specification of the present invention without particular mention are based on weights, and as an example, a unit of % or ratio refers to a wt % or a weight ratio.

In addition, in the specification of the present invention, the expression “comprise” is an open-ended description having a meaning equivalent to the expression such as “provided”, “contain”, “have”, or “is/are characterized”, and does not exclude elements, materials or processes which are not further listed. In addition, the expression “substantially consisting of . . . ” means that other elements, materials, or processes which are not listed together with specified elements, materials, or processes may be present in an amount which does not have a significant influence which is unacceptable in at least one basic and novel technical idea of the invention. In addition the expression “consisting of” means that only the described elements, materials, or processes are present.

The term “component”, “composition”, “composition of a compound”, “compound”, “drug”, “pharmaceutical activator”, “activator”, “cure”, “therapeutic method”, “treatment”, or “medicine” used in the specification of the present invention is interchangeably used for meaning a compound or compound(s) or a composition of a material inducing a desired pharmaceutical and/or physiological effect by a topical and/or systemic action when administered to a subject (human or animal).

The term “treatment” or “therapeutic method” (as well as different forms thereof) used in the specification of the present invention includes preventive (e.g., preventive treatment), curative, or mitigating treatments. The term “treating” used in the present application includes alleviating or decreasing at least one harmful or negative effect or symptom of states, diseases, or disorders.

In the present invention, a sample or a specimen represents a subject to be analyzed, and is used in the same sense throughout the specification.

Hereinafter, the present invention will be described in more detail.

The pharmaceutical composition for improving cognitive ability or enhancing memory according to the present invention includes N-palmitoyl serinol as an effective component.

The N-palmitoyl serinol (trade name: Neuromide) may be usually obtained by reacting palmitoyl chloride and 2-amino-1,3-propanediol, but is not limited thereto.

The pharmaceutical composition may be formulated into common preparations in the pharmaceutical field, for example, preparations for oral administration such as tablets, pills, hard/soft capsules, liquid, suspension, emulsifier, syrup, granules, and elixirs or preparations for parenteral administration such as sterile aqueous or oily solvents for intravenous, subcutaneous, sublingual, and intramuscular administration.

A pharmaceutically acceptable carrier which may be used in the pharmaceutical composition of the present invention is commonly used in formulation, and includes lactose, dextrose, sucrose, sorbitol, mannitol, starch, acacia gum, calcium phosphate, alginate, gelatin, calcium silicate, microcrystalline cellulose, polyvinylpyrrolidone, cellulose, water, syrup, methyl cellulose, methyl hydroxybenzoate, propyl hydroxybenzoate, talc, magnesium stearate, and/or mineral oil, and the like, but is not limited thereto.

An excipient which may be used in the pharmaceutical composition of the present invention may be a sweetener, a binder, a solubilizer, a solubilizing aid, a wetting agent, an emulsifier, an isotonic agent, an adsorbent, a disintegrant, an antioxidant, a preservative, a lubricant, a filler, a fragrance, or the like, and a ratio and properties of the excipient may be determined by solubility and chemical properties of a selected tablet, a selected administration route, and standard pharmaceutical practice. An example of the excipient may include lactose, dextrose, sucrose, mannitol, sorbitol, cellulose, glycine, silica, talc, stearic acid, stearin, a magnesium stearate salt, glyceryl monostearate, a magnesium aluminum silicate salt, starch, gelatin, tragacanth rubber, alginic acid, sodium alginate, methyl cellulose, sodium carboxymethyl cellulose, agar, water, ethanol, polyethylene glycol, polyvinylpyrrolidone, sodium chloride, potassium chloride, orange essence, strawberry essence, vanilla flavor, or the like.

In addition, the pharmaceutical composition of the present invention may be formulated into a parenteral administration form, and in this case, intravenous administration, intraperitoneal administration, intramuscular administration, subcutaneous administration, topical administration, or the like may be used, but is not limited thereto. Here, in order to formulate the composition into a formulation for parenteral administration, the pharmaceutical composition may be prepared by mixing an effective component with a stabilizer or a buffer in water to produce a solution or a suspension and producing the solution or the suspension into a unit dosage form of an ampule or a vial.

In addition, the pharmaceutical composition of the present invention may be sterilized, or further include an adjuvant such as a preservative, a stabilizer, a hydrating agent or an emulsifying accelerator, a salt for regulating osmotic pressure, and/or a buffer, and other therapeutically useful materials, and may be formulated according to a conventional method of mixing, granulating or coating.

In addition, a dosage of the effective component for mammals including humans in the pharmaceutical composition according to the present invention may vary depending on age, weight, gender, dosage form, health condition, and a degree of disease of a patient. Generally, an effective amount of 0.001 to 500 mg/kg (body weight), preferably 0.01 to 300 mg/kg (body weight) per day may be included in the pharmaceutical composition, and the pharmaceutical composition may be divided and administered once or twice per day via an oral or parenteral route. However, since the amount may be increased or decreased depending on an administration route, severity of the disease, gender, weight, age, and the like, the administration amount does not limit the scope of the present invention in any way.

In the pharmaceutical composition for improving cognitive ability or enhancing memory according to an exemplary embodiment of the present invention, the pharmaceutical composition may be for improving or treating neurodegenerative brain diseases or neuroinflammatory diseases.

In the pharmaceutical composition for improving cognitive ability or enhancing memory according to an exemplary embodiment of the present invention, the disease may be selected from the group consisting of autism, Alzheimer's disease, Parkinson's disease, peripheral neuropathy, amyotrophic lateral sclerosis, ischaemia, stroke, and multiple sclerosis.

Alzheimer's disease (AD) is a chronic and progressive neurodegenerative brain disease, and causes memory loss, disorientation, cognitive dysfunction, and behavioral disorders. The Alzheimer's disease is related to loss of choline in the basal forebrain and cerebral cortex. Since lack of cholinergic neurotransmission plays a decisive role in decline in learning and memory of an Alzheimer disease patient, it has been reported that it may be effective for treating Alzheimer's disease to inhibit cholinesterase to activate the function of choline. A synthetic acetylcholine esterase inhibitor including Tacrine, Donepezil, Rivastigmine, and Galanthamine has been used as a therapeutic agent for Alzheimer's disease. However, since it is known that these drugs cause side effects such as insufficient activity, hepatotoxicity, and gastrointestinal disorders, improvement thereon is demanded.

Besides, it is known that the ultimate cause of neurodegenerative brain diseases represented by Parkinson's disease, Alzheimer's disease, dementia, stroke, and the like is cell death leading to neuronal damage and loss. As the main cause of neuronal death of neurodegenerative brain diseases, active oxygen is pointed out. Active oxygen refers to oxygen having strong oxidizing power which is produced in several metabolic processes as oxygen entering the body is used in an oxidation process and attacks living tissues and damages cells. The active oxygen irreversibly damages DNA in living cells, cell-constituting proteins, lipids, and the like (Valko et al., Free radicals and antioxidants in normal physiological functions and human disease. Int J Biochem Cell Biol. 39(2007), 44-84). In particular, when a large amount of active oxygen is produced in brain cells, oxidative stress causes change in the structure and the function of mitochondria in cells, thereby causing neurological brain diseases such as Parkinson disease and Alzheimer's disease (Knott et al., Mitochondrial fragmentation in neurodegeneration. Nat Rev Neurosci., 9(2008), 505-18).

A specific formulation of the pharmaceutical composition for improving cognitive ability or enhancing memory according to the present invention may be in the form of a multi-lamella emulsion or orthorhombic crystalline phase, but is not limited thereto.

The health functional food for improving cognitive ability or enhancing memory according to the present invention includes N-palmitoyl serinol as an effective component.

The health functional food for improving cognitive ability or enhancing memory or the health functional food including the composition of the present invention may include a material having a trade name of Neuromide, which is N-palmitoyl serinol as an actual component name, as an effective component.

In the present invention, “health functional food” refers to a natural or processed product containing one or more nutrients, and preferably, refers to a food group to which value is added so that the function of a corresponding food works and is expressed for a specific purpose, using a method such as physical, biochemical, and biotechnological methods, or a food which is designed and processed so that a body control function related to biological defense rhythm control, disease prevention and recovery, and the like of a food composition is sufficiently expressed in the living body. The health functional food may include a food auxiliary additive which is acceptable in food science, and may further include appropriate carrier, excipient, and diluent which are commonly used in the manufacture of the health functional food.

An example of the health functional food to which the effective component of the present invention may be added includes various foods, beverages, chewing gum, tea, vitamin complexes, and the like. Further, it includes special nutritional foods (e.g., milk formula, infant/baby food, etc.), health supplement, confectionery (e.g., snacks), dairy products (e.g., fermented milk, cheese, etc.), other processed foods, beverages (e.g., fruit, vegetable beverages, soy milk, fermented beverages, etc.), and the like, but is not limited thereto. The food, the beverage, or the food additive described above may be manufactured by a common manufacturing method.

The health functional food of the present invention may contain various nutritional supplements, vitamins, minerals (electrolyte), flavors such as synthetic flavors and natural flavors, colorants and enhancers (cheese, chocolate, etc.), pectic acid and salts thereof, alginic acid and salts thereof, organic acids, protective colloid thickeners, pH regulators, stabilizers, preservatives, glycerin, water, carbonation agent used in carbonated drink, and the like. These components may be used alone or in combination.

As such, the health functional food of the present invention may have various formulations as described above, and in particular, may have any one formulation in powder, granules, tablets, capsules, beverages, and the like, but is not limited thereto.

As an example, a specific formulation of the health functional food including the composition for improving cognitive ability or enhancing memory according to the present invention may be in the form of a multi-lamella emulsion or orthorhombic crystalline phase, but is not limited thereto.

The composite pharmaceutical composition for improving cognitive ability or enhancing memory according to the present invention includes N-palmitoyl serinol and an active material for improving cognitive ability or enhancing memory.

In the composite pharmaceutical composition for improving cognitive ability or enhancing memory according to an exemplary embodiment of the present invention, the composite pharmaceutical composition may include first particles including N-palmitoyl serinol; and second particles including an active material for improving cognitive ability or enhancing memory.

The oil phase may be any one or more selected from the group consisting of silicon oil, ester-based oil, hydrocarbon-based oil, propylene glycol monocaprylate (Capryol 90), propylene glycol dicaprylocaprate (Labrafac PG), oleoyl macrogol-6 glyceride (Labrafil M1944 CS), lauoryl macrogol-6 glyceride (Labrafil M2130 CS), linoleoyl macrogol-6 glyceride (Labrafil M2125 CS), medium chain triglyceride (Labrafac), oleic acid, stearic acid, glyceryl behenate (Compritol 888), glycerol monostearate, and castor oil, but is not limited thereto, and is not limited as long as it is used for improving cognitive ability or enhancing memory using the pharmaceutical composition or the composite pharmaceutical composition of the present invention and has no problem such as causing toxicity.

In the composite pharmaceutical composition for improving cognitive ability or enhancing memory according to an exemplary embodiment of the present invention, the composite pharmaceutical composition may further include a sterol-based compound, a higher fatty acid, and a higher fatty acid alcohol, and may form a very stable crystal lamella micro-encapsulation system (MES) or orthorhombic crystalline phase therefrom, but the scope of the present invention is not limited thereto.

As an example, the sterol-based compound may be one or more selected from cholesterol, phytosterol, 3b-[N-(N′,N′-dimethylaminoethane)-cabamyl]cholesterol (DC-Chol), stigmasterol, campesterol, sitosterol, ergosterol, lanosterol, dinosterol, gorgosterol, avenasterol, saringosterol, fucosterol, cholesteryl hemisuccinate, cholesteryl benzoate, cholesteryl oleate, cholesteryl oleyl carbonate, cholesteryl isostearate, cholesteryl linoleate, cholesteryl acetate, cholesteryl palmitate, cholesteryl stearate, cholesteryl chloride, cholesteryl nonanoate, cholesteryl arachidonate, and the like, but is not limited thereto. In addition, the higher fatty acid may be, as an example, a higher fatty acid having 10 to 25 carbon atoms, and specifically, may be one or more selected from oleic acid, linoleic acid, behenic acid, arachidonic acid, stearic acid, palmitic acid, myristic acid, lauric acid, and the like. In addition, the higher fatty acid alcohol may be, as an example, a higher fatty acid alcohol having 10 to 25 carbon atoms, and specifically, may be one or more selected from cetearyl alcohol, stearyl alcohol, cetostearyl alcohol, behenyl alcohol, and the like, but the scope of the present invention is not limited thereto.

In the pharmaceutical composition for improving cognitive ability or enhancing memory according to an exemplary embodiment of the present invention, 10 to 1000 parts by weight, preferably 100 to 900 parts by weight, and more preferably 300 to 500 parts by weight of the sterol-based compound, 100 to 3000 parts by weight, preferably 100 to 2000 parts by weight, and more preferably 100 to 1000 parts by weight of the higher fatty acid, 100 to 5000 parts by weight, preferably 150 to 2500 parts by weight, and more preferably 1500 to 2000 parts by weight of the higher fatty acid alcohol, and 1 to 500 parts by weight, preferably 1 to 300 parts by weight, and more preferably 1 to 100 parts by weight of the active material, with respect to 100 parts by weight of the N-palmitoyl serinol may be mixed, but the present invention is not limited thereto, and within the range, an effect of improving cognitive ability or enhancing memory according to an effect of combining with the active material may be improved, which is thus preferred.

In the pharmaceutical composition for improving cognitive ability or enhancing memory according to an exemplary embodiment of the present invention, the active material may be any one or more selected from the group consisting of Donepezil, Rivastigmine, and Galantamine, but is not limited thereto, and any material having an effect of improving cognitive ability or enhancing memory may be generally introduced and used. Among the materials, Donepezil is well known as a drug for treating Alzheimer's disease, and it was confirmed that the composite pharmaceutical composition according to an exemplary embodiment of the present invention shows a synergistic effect in improving cognitive ability or enhancing memory.

The method of preparing a composite pharmaceutical composition for improving cognitive ability or enhancing memory according to the present invention includes: preparing first particles including N-palmitoyl serinol; and preparing second particles including an active material for improving cognitive ability or enhancing memory.

In the method of preparing a composite pharmaceutical composition for improving cognitive ability or enhancing memory according to an exemplary embodiment of the present invention, the disease related to cognitive impairment or memory loss may be selected from the group consisting of autism, Alzheimer's disease, Parkinson's disease, peripheral neuropathy, amyotrophic lateral sclerosis, ischaemia, stroke, and multiple sclerosis.

A mixing ratio of the first particles and second particles is not particularly limited, but when the second particles are, as an example, a material selected from synthetic aceteylcholine esterase inhibitors including Tacrine, Donepezil, Revastigmine, and Galanthamine, as an example, may be mixed at a weight ratio of 100:0.1 to 2, preferably 100:0.3 to 1.7, more preferably 100:0.5 to 1.5, and by mixing particles at the ratio, the insufficient activity and the side effects such as hepatotoxicity and gastrointestinal disorders of the second particles may be blocked, which is thus highly preferred.

The administration method for improving cognitive ability and memory decline according to the present invention includes administrating a pharmaceutical composition for improving cognitive ability or enhancing memory including N-palmitoyl serinol as an effective component to a subject.

In the administration method for improving cognitive ability or memory decline according to an exemplary embodiment of the present invention, a dose of the pharmaceutical composition for improving cognitive ability or enhancing memory administered to the subject may be 1 to 200 mg/kg, preferably 10 to 170 mg/kg, and more preferably 25 to 140 mg/kg, based on the weight of the subject, but is not limited thereto, and within the range, the effect of improving cognitive ability and enhancing memory may be further improved, which is thus preferred.

In the administration method for improving cognitive ability or memory decline according to an exemplary embodiment of the present invention, an administration interval of the pharmaceutical composition for improving cognitive ability and enhancing memory administered to the subject may be 12 hours or more and 240 hours or less, preferably 24 hours or more and 240 hours or less, and more preferably 120 hours or more and 240 hours or less, but is not limited thereto, and the administration interval may be adjusted for improving the effect of improving cognitive ability and enhancing memory.

In the administration method for improving cognitive ability or memory decline according to an exemplary embodiment of the present invention, the administration method may include oral administration, subcutaneous injection, and subcutaneous application, but is not limited thereto, and the health food of the present invention may be administered by oral administration and the composite pharmaceutical composition may be administered regardless of the administration method, but an effect of combining drugs by a method by oral administration or subcutaneous injection is most significant and preferred.

In the administration method, the administration is performed in a form such as subcutaneous application, the pharmaceutical composition for improving cognitive ability or enhancing memory of the present invention may further include a component included a common cosmetic material, and thus, administration by application by a common formulation may be performed, of course.

In the subcutaneous application, the pharmaceutical composition may be, though it is not limited, any one formulation selected from the group consisting of solution, suspension, emulsion, paste, gel, cream, lotion, powder, soap, oil, powder foundation, emulsion foundation, wax foundation, and spray, and components commonly used in cosmetics may be included in addition to the effective component of the present invention, and for example, a common adjuvant such as antioxidants, stabilizers, solubilizer, vitamins, pigments, or flavoring, and carriers may be included.

Meanwhile, the composition may be produced into any formulation commonly prepared in the art, and for example, may be formulated into solution, suspension, emulsion, paste, gel, cream, lotion, powder, soap, surfactant-containing cleansing, oil, powder foundation, emulsion foundation, wax foundation, spray, and the like. However, it is not necessarily limited thereto. More specifically, it may be produced into a formulation of soft lotion, astringent lotion, nourishing lotion, nourishing cream, massage cream, essence, pack, spray, or powder.

When the formulation of the composition is paste, cream, or gel, animal oil, vegetable oil, wax, paraffin, starch, tragacanth, cellulose derivatives, polyethylene glycol, silicone, bentonite, silica, talc, zinc oxide, or the like may be used as a carrier component.

In addition, when the formulation of the composition is powder or spray, lactose, talc, silica, aluminum hydroxide, calcium silicate, or polyamide powder may be used as a carrier component, and in particular, when spray is used, a propellant such as chlorofluorohydrocarbon, propane/butane, or dimethyl ether may be further included.

In addition, the formulation of the composition is solution or emulsion, solvents, solubilizers, or emulsifiers are used as a carrier component, and for example, water, ethanol, isopropanol, ethyl carbonate, ethyl acetate, benzyl alcohol, benzyl benzoate, propylene glycol, 1,3-butylglycol oil, glycerol aliphatic ester, polyethylene glycol, or fatty acid ester of sorbitan may be used.

In addition, the formulation of the composition is suspension, liquid diluents such as water, ethanol, or propylene glycol, suspending agents such as ethoxylated isosterayl alcohol, polyoxyethylene sorbitol ester, and polyoxyethylene sorbitan ester, microcrystalline cellulose, aluminum metahydroxide, bentonite, agar, or tragacanth, and the like may be used as a carrier component.

The composition may further include sterol-based compounds, higher fatty acids and higher fatty acid alcohols, thereby forming a very stable crystal lamella micro-encapsulation system (MES). As an example, the sterol-based compound may be one or more selected from cholesterol, phytosterol, 3b-[N-(N′,N′-dimethylaminoethane)-cabamyl]cholesterol (DC-Chol), stigmasterol, campesterol, sitosterol, ergosterol, lanosterol, dinosterol, gorgosterol, avenasterol, saringosterol, fucosterol, cholesteryl hemisuccinate, cholesteryl benzoate, cholesteryl oleate, cholesteryl oleyl carbonate, cholesteryl isostearate, cholesteryl linoleate, cholesteryl acetate, cholesteryl palmitate, cholesteryl stearate, cholesteryl chloride, cholesteryl nonanoate, cholesteryl arachidonate, and the like, but is not limited thereto. In addition, the higher fatty acid may be, as an example, a higher fatty acid having 10 to 25 carbon atoms, and specifically, may be one or more selected from oleic acid, linoleic acid, behenic acid, arachidonic acid, stearic acid, palmitic acid, myristic acid, lauric acid, and the like. In addition, the higher fatty acid alcohol may be, as an example, a higher fatty acid alcohol having 10 to 25 carbon atoms, and specifically, may be one or more selected from cetearyl alcohol, stearyl alcohol, cetostearyl alcohol, behenyl alcohol, and the like, but the scope of the present invention is not limited thereto.

In addition, the composition may further include ceramide for enhancing stability of a liquid crystal. As an example, the ceramide may be selected from natural ceramide, synthetic ceramide, and pseudo-ceramide, and specifically, may be one or more selected from ceramide 1, ceramide 3, ceramide 3B, ceramide 4, ceramide 6, myristoyl/palmitoyl oxostearamide/arachamide MEA (product name: PC-9S), hydroxypropylbispalmitamide MEA (product name: PC-104), hydroxypropylbispalmitamide MEA (product name: PC-102), and dihydroxyisopropylpamoyl palmamide, and the like, but is not limited thereto.

Hereinafter, the present invention will be described in detail by way of example. The examples are only for describing the present invention in more detail, and the scope of the present invention is not limited to the following examples.

### Materials, Reagents, and Equipment of Experiment

An ICR mouse CrljOri:CD1, male, 7 weeks old, 29.7-33.8 g: ORIENT BIO., the Republic of Korea
Product name, Neuromide®: component name, N-palmitoyl serinol: Kyung-In Synthetic Corporation, the Republic of Korea
Component name, glyceryl stearate: purchased in the Republic of Korea and used.
CMC(carboxymethyl cellulose): Sigma-Aldrich, MO

CMC was dissolved in sterile tap water and prepared to be 0.5% (w/c)
Donepezil hydrochloride: Sigma-Aldrich, MO
Other reagents and instruments for measurement were purchased in the Republic of Korea and used.

### Preparation Example

### Excipient

40 mg of glyceryl stearate was measured with an electronic scale and injected into a disposable tube.

Carboxymethyl cellulose (CMC) was mixed with sterile tap water and prepared to be 0.5% (w/v), a small amount of the mixture was injected into the tube, homogenization was performed by vortexing, the mixture was added up to 10 mL to the tube, homogenization was performed by vortexing again, and then the mixture was used in the test.

### Test Material (Neuromide)

16 mg, 40 mg, and 100 mg of Neuromide were measured with an electronic scale and injected into a disposable tube, respectively, and 40 mg of glyceryl stearate was added. Thereafter, a small amount of 0.5% (w/v) carboxymethyl cellulose (CMC) was added, respectively, homogenization was performed by vortexing, the mixture was added up to 10 mL to each tube, and each tube was homogenized by vortexing again and then used in the test.

### Comparative Material (Donepezil Hydrochloride)

1 g of Donepezil hydrochloride was measured with an electronic scale and injected into a disposable tube. Thereafter, a small amount of 0.5% (w/v) carboxymethyl cellulose (CMC) was added thereto, homogenization was performed by vortexing, the mixture was added up to 10 mL to the tube, homogenization was performed by vortexing again, and the mixture was used in the test.

### Composite Material for Combination Treatment (Neuromide+Donepezil Hydrochloride)

100 mg of Neuromide was measured with an electronic scale, 1 mg of Donepezil hydrochloride was measured with an electronic scale, and they were injected into the same tube.

40 mg of glyceryl stearate was measured by an electronic scale and injected into the tube, homogenization was performed by vortexing, 0.5% (w/v) carboxymethyl cellulose (CMC) was added up to 10 mL to the tube, homogenization was performed by vortexing again, and then the mixture was used in the test.

### [Test Example 1] Quarantine/Purification and Selection of Mouse to be Experimented On

### Quarantine/Purification of Mouse

7 week old 53 ICR mice were brought in and visually inspected, and the weight of each was measured.

Thereafter, the mice went through a purification period of 7 days, and it was observed whether there was a general symptom for 5 days of the period.

On the date the purification period for 7 days ended, the weights were measured, and change in general symptom and weight was confirmed to evaluate the health condition of the mice.

As a result, there was no mouse showing an abnormal condition during quarantine and purification.

### Marking and Selection of Mouse Object

During purification, an object mark was made on the tail of the brought-in mouse using a red oil pen, and an object identification card was attached to a breeding box which was then managed.

49 mice which had no abnormality in terms of health and were close to average weight during purification were selected and divided into 7 experimental groups of 7 mice each so that the average weight of each group was equivalent, and were used in the test.

After completing the purification, an object mark was made on the tail of the selected mouse using a blue oil pen during the test period, and an object identification card was also attached to the breeding box which was then managed.

The object which was not selected as the test subject was separated from the experimental group, and was euthanized under CO₂ gas anesthesia.

### [Test Example 2] Mouse Breeding Environment

Mice were bred in an animal room No. SPF-2606 of the laboratory animal center of Osong Medical Innovation Foundation in Chungbuk.

The box used in breeding was a box having a size of 200×310×150 mm (width×breadth×height) made of polysulfonate, and 2-3 objects were housed per each breeding box.

The breeding room was maintained at room temperature of 20.0 to 24.0° C. at a relative humidity of 40.0 to 60.0%.

For a pleasant bleeding environment, automatic ventilation was performed 10 to 15 times per hour, and a dark-light cycle was adjusted using a light daily for 12 hours each from 8:00 am to 8:00 pm and from 8:00 pm to 8:00 am the next day. A bright light source having a lighting illuminance of 500 Lux or more was used.

The box, the feeder, and the drinking bottle used in breeding were exchanged at a frequency of twice a week to maintain the pleasant environment.

The feed used in breeding was a product imported and supplied by WOOJUNGBIO Inc. (Korea), which was +40 RMM (SAFE complete care competence, France), and the product in the form of solid feed was injected into the feeder to allow the mouse objects to consume it freely.

As water, sterile water was put in a drinking bottle, which was placed in the breeding box to allow the mouse objects to drink it freely.

### [Test Example 3] Drug Administration and Observation

Each material prepared in the preparation example was prepared so that each may be orally administered to 7 experimental groups in the following and Table 1.

In the following Table 1, only the excipient prepared in the preparation examples was orally administered to a normal control group and a negative control group, respectively, scopolamine was administered to the remaining experimental groups (G2-G7) other than the normal control group (G1) in the behavioral experiment of Test Example 4 to cause cognitive function decline and a memory impairment effect, and then the behavior was observed.

TABLE 1
Drug administration experimental group and experimental
condition

| | | | | Administration | Number |
|---|---|---|---|---|---|
| | Experimental group | | | liquid | of |
| | Serial | | Administration | amount | administration |
| | number | Characteristics | dose (mg/kg) | (mL/kg) | (times) |
| Comparative | G1 | Normal control group | — | 10 | 42 |
| Example 1 | | | | | |
| Comparative | G2 | Negative control group | — | 10 | 42 |
| Example 2 | | | | | |
| Example 1 | G3 | Experimental material low | 16 | 10 | 42 |
| | | dose administration group | | | |
| Example 2 | G4 | Experimental material | 40ª | 10 | 42 |
| | | medium dose | | | |
| | | administration group | | | |
| Example 3 | G5 | Experimental material | 100 | 10 | 42 |
| | | high dose administration | | | |
| | | group | | | |
| Comparative | G6 | Comparative material | 1^{b} | 10 | 42 |
| Example 3 | | administration group | | | |
| Example 4 | G7 | Combined administration | Mixture of test | 10 | 42 |
| | | group of test material | material 100: | | |
| | | and comparative material | comparative | | |
| | | | material 1 | | |

^{a}Value obtained by converting an adult daily intake dose of 200 mg/60 kg into a dose administered to mice: 40 mg/kg.
^{b}Value obtained by converting a clinical application dose of 5 mg/kg into a dose administered to mice: 1 mg/kg.

On the day of the experiment, general symptoms such as appearance, behavior, and excrement of the object for each experimental group were observed, and it was also confirmed whether dead objects occurred after administration.

For all objects of each experimental group, when drug administration started, the body weight was measured twice a week from the start of administration until the end of the experiment, and was measured on the date of the final autopsy.

### [Test Example 4] Mouse Passive Avoidance Test and Analysis

### Passive Avoidance Test

After the test material (drug) of Test Example 3 was administered to each experimental group during the test, a passive avoidance test was performed, and change in mouse behavior was observed.

After each of the test materials was administered to each experimental group of Table 1 of Test Example 3, the passive avoidance test was performed on day 25 by the following procedure:
a) The object was put into an unlit white room of a conditioning box for the passive avoidance test, and then was adapted for 60 seconds.
b) A guillotine door was opened simultaneously with turning on a LED light of the white room, and the object was allowed to explore a dark room for 180 seconds for adaptation.
c) On the next day (day 26) also, the above process was performed and repeated once more, and the object was adapted so that it took around 30 seconds to enter the dark room after turning on the LED light.
d) After administering the drug 1 hour before learning on day 27, 4 mL/kg of Scopolamine which was dissolved at 0.25 mg/mL in a 0.9% saline solution was intraperitoneally administered to the objects of the remaining experimental groups other than the normal control group (G1), respectively, after 30 minutes, and the objects were allowed to stand for 30 minutes in the breeding box.
e) 30 minutes after administering Scopolamine, the objects were put into the unlit white room, the guillotine door was opened simultaneously with turning on the LED light after 10 seconds. When all of the four legs of the objects entered to the dark room, the guillotine door was closed, the time spent (latency time) was measured with a timer, and electrical stimulation was given at 0.5 mA for 3 seconds.

### Treatment of Tissue of Object After End of Test

On the next day of the end of the passive avoidance test, the test material was administered, and the objects were allowed to stand in a home cage for 30 minutes. Subsequently, Scopolamine was intraperitoneally administered at a dose of 1 mg/kg, and then the objects were allowed to stand in a home cage for 30 minutes.

Respiratory anesthesia was given with isofluorane, blood was taken from an abdominal vein, and euthanasia was performed by releasing blood. Each blood taken was placed in a serum separation tube, and was centrifuged at 4° C. at 3,000 rpm for 15 minutes. A supernatant serum was separated and stored at −70° C. until analysis.

A brain tissue was separately separated and stored at −70° C. until analysis.

### Measurement of Acetylcholine and Acetylcholine Esterase (AchE) Activities in Brain Tissue

A choline assay buffer (Biovision, CA, USA) was added to a hippocampal tissue of the separated brain, crushed, and then centrifuged to obtain a supernatant liquid, which was used in the test.

The amount of acetylcholine in the supernatant liquid was measured using a choline/acetylcholine quantification kit (Biovision, CA, USA).

Acetylcholine esterase (AchE) activity was measured with a supernatant liquid obtained by adding a tissue protein extraction reagent (Biovision, CA, USA) to a forebrain tissue, crushing it, and centrifuging it, using an acetylcholinesterase activity assay kit (Biovision, CA, USA).

### Measurement of Inflammatory Cytokines TNF-α and Il-1β in Brain Tissue

Inflammatory cytokine in a brain tissue was measured with a supernatant liquid obtained by adding a tissue protein extraction reagent (Biovision, CA, USA) to a brain tissue, crushing it, and centrifuging it, using an ELISA Kit (R&D System, MN, USA).

All data obtained in the test was assayed using SPSS Ver. 21.0 (SPSS Inc., USA).

When a significance was observed in an independent sample T-test, the independent sample T-test for confirming a test group having a significant difference from a control group was performed (significance level single measurement: 5%).

### Measurement of Ceramide Content in Brain Tissue

Two types of forebrain parts of experimental groups G1, G2, and G5 in the brain tissues separated above were secured, respectively.

The forebrain 1-2 and 1-3 of G1 showed protein contents of 201.3312 and 185,3896 μg, respectively, the forebrain 2-2 and 2-3 of G2 showed protein contents of 198.3044 and 229.1784 μg, respectively, and the forebrain 5-2 and 5-3 of G5 to which 100 mg/kg of Neuromide was administered showed protein contents of 180.9504 and 196.0848 μg, respectively.

The ceramide contents were analyzed therefrom, and the average and the deviation thereof were calculated.

At this time, the measurement of the ceramide content was performed by confirmation and quantitative method of ceramide commonly known to a person skilled in the art.

### Measurement of Sphingoid Base Content in Brain Tissue

Two types of forebrain parts of the experimental groups G1, G2, and G5 in the separated brain tissue were secured, respectively, the contents of sphingoid base were analyzed, and the average and the deviation were calculated.

At this time, the measurement of contents of sphingosine (SPN), sphinganine (Sa), sphingosine-1-phosphate (S1P), and sphinganine-1-phosphate (Sa1P) which are sphingoid bases was performed by confirmation and quantitative method of sphingoid bases commonly known to a person skilled in the art.

The test results are as follows:

1) Passive Avoidance Test

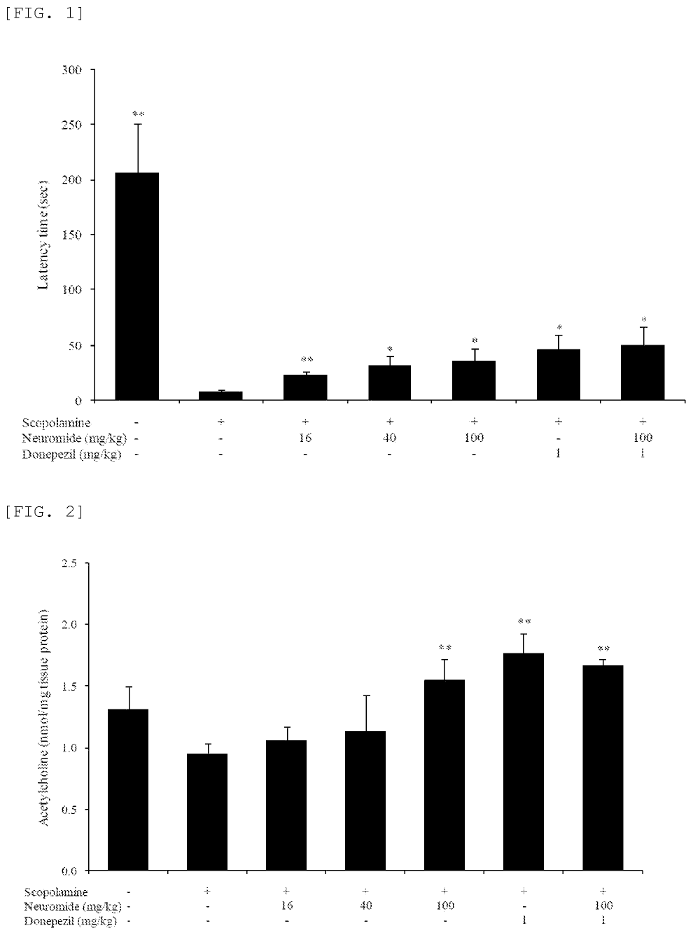

The results of performing the passive avoidance test by the method of Test Example 4 are shown in the following Table 2 and FIG. 1.

TABLE 2
Average data of time taken in passive avoidance test

| | | | | Passive |
|---|---|---|---|---|
| | Experimental group | | | avoidance test |
| | Serial | | Administration | Time taken |
| | number | Characteristics | dose (mg/kg) | (sec) |
| Comparative | G1 | Normal control group | — | 205.9 ± 45.2 ** |
| Example 1 | | | | |
| Comparative | G2 | Negative control group | — | 7 ± 2.3 |
| Example 2 | | | | |
| Example 1 | G3 | Neuromide | 16 | 22.3 ± 3.6 |
| Example 2 | G4 | Neuromide | 40 | 31 ± 8.9 * |
| Example 3 | G5 | Neuromide | 100 | 35.7 ± 11.5 * |
| Comparative | G6 | Donepezil | 1 | 45.6 ± 14 * |
| Example 3 | | | | |
| Example 4 | G7 | Combined administration | Mixture of | 50.1 ± 15.8 * |
| | | group of Neuromide and Donepezil | Neuromide 100: | |
| | | | Donepezil 1 | |

Data is expressed as a format of mean ± standard error.
Significant difference from the negative control group (G2) by independent t-test:
* p < 0.05,
** p < 0.01

As confirmed therefrom, the time taken in the negative control group was 7 seconds on average, which was statistically significantly decreased as compared with 205.9 seconds in the normal control group, and it was confirmed therefrom that memory was decreased by scopolamine.

In addition, it was confirmed that the time taken in the single administration group of the test material, Neuromide (Examples 1 to 3) was 22.3 seconds, 31 seconds, and 35.7 seconds on average, respectively, which was statistically significantly increased as compared with the negative control group, and the time taken in the single administration group of the comparative material, Donepezil (Comparative Example 3) and a combined administration group of the test material and the comparative material (Example 4) was 45.6 seconds and 50.1 seconds on average, respectively, which were statistically significantly increased as compared with the negative comparative group.

2) Acetylcholine

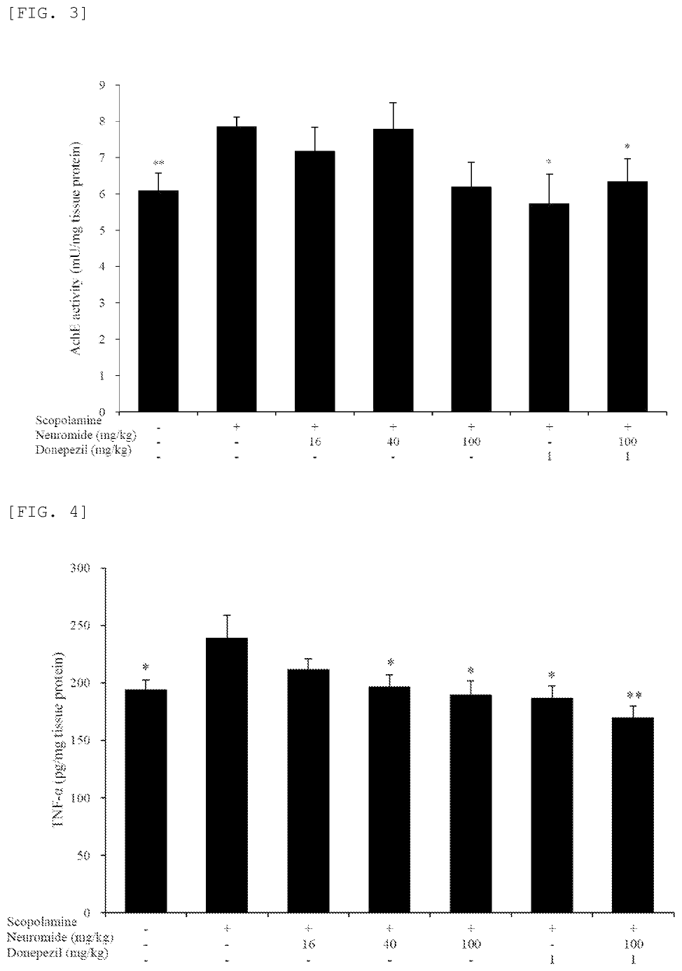

In order to evaluate the effect of the test material on memory improvement, acetylcholine in the hippocampus area of the brain was measured after completing the passive avoidance test, and the results are shown in the following Table 3 and FIG. 2:

TABLE 3

| | Experimental group | | | Acetylcholine |
|---|---|---|---|---|
| | Serial | | Administration | Nmol/mg |
| | number | Characteristics | dose (mg/kg) | protein |
| Comparative | G1 | Normal | — | 1.3 ± 0.2 |
| Example 1 | | control group | | |
| Comparative | G2 | Negative | — | 0.9 ± 0.1 |
| Example 2 | | control group | | |
| Example 1 | G3 | Neuromide | 16 | 1.1 ± 0.1 |
| Example 2 | G4 | Neuromide | 40 | 1.1 ± 0.3 |
| Example 3 | G5 | Neuromide | 100 | 1.5 ± 0.2 ** |
| Comparative | G6 | Donepezil | 1 | 1.8 ± 0.2 ** |
| Example 3 | | | | |
| Example 4 | G7 | Combined | Mixture of | 1.7 ± 0.1 ** |
| | | administration group | Neuromide 100: | |
| | | of Neuromide and | Donepezil 1 | |
| | | Donepezil | | |

Data is expressed as a format of mean ± standard error.
Significant difference from the negative control group (G2) by independent t-test:
** p < 0.01

As confirmed therefrom, the amount of acetylcholine of the hippocampus in the negative control group having learning ability and memory decreased by administrating scopolamine was 0.9 nmol/mg protein on average, which was decreased as compared with 1.3 nmol/mg protein of the normal control group.

However, the amounts of acetylcholine in Examples 1 to 3 to which Neuromide as the test material was administered in different amounts were 1.1 nmol/mg protein, 1.1 nmol/mg protein, and 1.5 nmol/mg protein on average, respectively, which were increased as compared with the negative control group, and it was confirmed that among them, the administration group of 100 mg/kg showed a statistically significant difference.

The amounts of acetylcholine of the administration group of 1 mg/kg of Donepezil as the comparative material (Comparative Example 3) and the combined administration group of the test material and the comparative material (Example 4) were 1.8 nmol/mg protein and 1.7 nmol/mg protein on average, respectively, which were increased as compared with the negative control group, and it was confirmed that a statistically significant difference was shown.

3) Acetylcholinesterase Activity

In order to evaluate the effect of the test material on memory improvement, acetylcholinesterase (AchE) activity in a forebrain area was measured after completing the passive avoidance test, and the results are shown in the following Table 4 and FIG. 3:

TABLE 4
Acetylcholinesterase activity

| | | | | Acetylcholine- |
|---|---|---|---|---|
| | Experimental group | | Administration | sterase (AchE) |
| | Serial | | dose | mU/mg tissue |
| | number | Characteristics | (mg/kg) | protein |
| Comparative | G1 | Normal | — | 6.09 ± 1.28 ** |
| Example 1 | | control group | | |
| Comparative | G2 | Negative | — | 7.85 ± 0.7 |
| Example 2 | | control group | | |
| Example 1 | G3 | Neuromide | 16 | 7.17 ± 1.75 |
| Example 2 | G4 | Neuromide | 40 | 7.79 ± 1.93 |
| Example 3 | G5 | Neuromide | 100 | 6.19 ± 1.84 * |
| Comparative | G6 | Donepezil | 1 | 5.73 ± 2.16 * |
| Example 3 | | | | |
| Example 4 | G7 | Combined | Mixture of | 6.33 ± 1.7 * |
| | | administration | Neuromide | |
| | | group of | 100: | |
| | | Neuromide and | Donepezil 1 | |
| | | Donepezil | | |

Data is expressed as a format of mean ± standard error.
Significant difference from the negative control group (G2) by independent t-test:
* p < 0.05,
** p < 0.01

Acetylcholinesterase (AchE) is an enzyme which decomposes the neurotransmitter acetylcholine, and when it is activated, signal transduction in nerves by acetylcholine is impaired.

As confirmed from the above results, the acetylcholinesterase activity of the negative control group is 7.85 mU/mg tissue protein on average, which was higher than 6.09 mU/mg tissue protein of the average of the normal control group.

In addition, it was confirmed that the acetylcholinesterase activity of the single administration group of the test material, Neuromide (Examples 1 to 3) was 7.17 mU/mg tissue protein, 7.79 mU/mg tissue protein, and 6.19 mU/mg tissue protein, respectively, which were decreased as compared with the negative control group, and among them, Example 3 was statistically significantly decreased.

It was confirmed that the acetylcholinesterase activity of the single administration group of the comparative material, Donepezil (Comparative Example 3) and the combined administration group of the test material and the comparative material (Example 4) was 5.73 mU/mg tissue protein and 6.33 mU/mg tissue protein, respectively, which were statistically significantly decreased as compared with the negative control group.

4) Inflammatory Cytokine

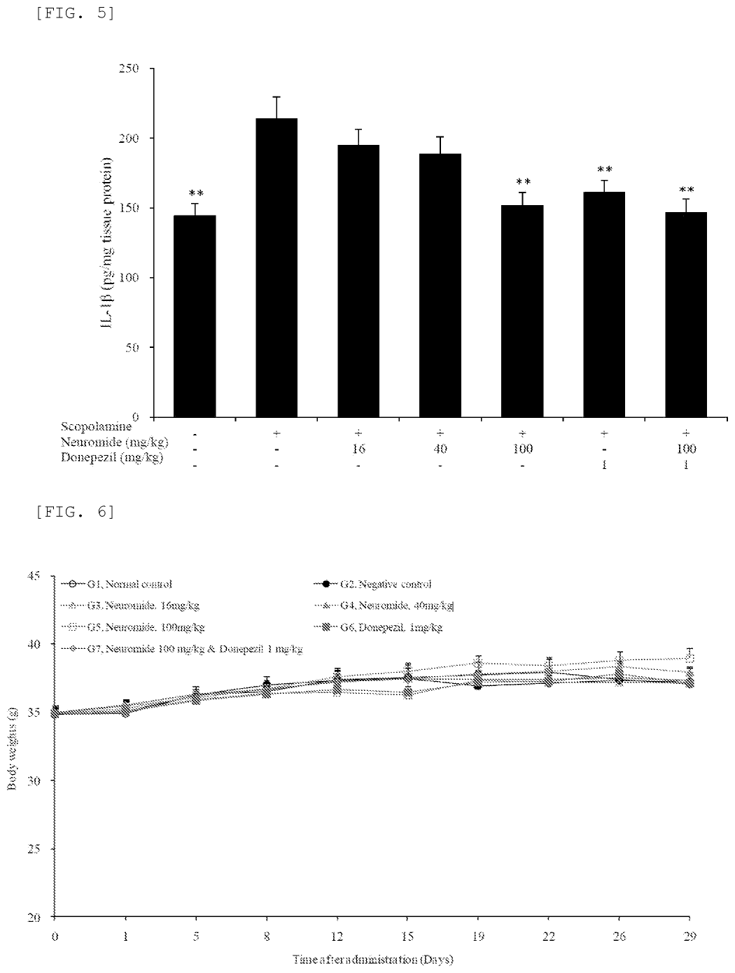

In order to evaluate the effect of the test material on the inflammatory cytokine, the amounts of TNF-α and Il-1β in the hippocampus area were measured after completing the passive avoidance test, and the results are shown in the following Table 5 and FIGS. 4 and 5:

TABLE 5
Inflammatory cytokine

| | Experimental group | | Administra- | TNF-α | IL-1β |
|---|---|---|---|---|---|
| | Serial | | tion dose | pg/mg tissue | pg/mg tissue |
| | number | Characteristics | (mg/kg) | protein | protein |
| Comparative | G1 | Normal control | — | 194 ± 22.2 * | 144.4 ± 22.8 ** |
| Example 1 | | group | | | |
| Comparative | G2 | Negative control | — | 239 ± 52 | 214 ± 41.4 |
| Example 2 | | group | | | |
| Example 1 | G3 | Neuromide | 16 | 211.5 ± 25.3 | 194.7 ± 30.6 |
| Example 2 | G4 | Neuromide | 40 | 196.2 ± 28.4 * | 188.6 ± 32.4 |
| Example 3 | G5 | Neuromide | 100 | 189.2 ± 33 * | 151.5 ± 24.9 ** |
| Comparative | G6 | Donepezil | 1 | 186.2 ± 30.3 * | 160.9 ± 23.2 ** |
| Example 3 | | | | | |
| Example 4 | G7 | Combined | Mixture of | | |
| | | administration | Neuromide | 169.8 ± 27.6 ** | 146.6 ± 25.9 ** |
| | | group of | 100: | | |
| | | Neuromide and | Donepezil | | |
| | | Donepezil | 1 | | |

Data is expressed as a format of mean ± standard error.
Significant difference from the negative control group (G2) by independent t-test:
* p < 0.05,
** p < 0.01

a. TNF-α

As confirmed therefrom, TNF-α of the negative control group was 239 pg/mg tissue protein, which was higher than 194 pg/mg tissue protein of the average of the normal control group.

In addition, it was confirmed that TNF-α of the single administration group of the test material, Neuromide (Examples 1 to 3) was 211.5 pg/mg tissue protein, 196.2 pg/mg tissue protein, and 189.2 pg/mg tissue protein on average, respectively, which were decreased as compared with the negative control group, and among them, Examples 2 and 3 were statistically significantly decreased as compared with the negative control group.

It was confirmed that TNF-α of the single administration group of the comparative material, Donepezil (Comparative Example 3) and the combined administration group of the test material and the comparative material (Example 4) was 186.2 pg/mg tissue protein and 169.8 pg/mg tissue protein on average, respectively, which were statistically significantly decreased as compared with the negative control group.

b. IL-1β

As confirmed therefrom, IL-1β of the negative control group was 214 pg/mg tissue protein, which was higher than 144.4 pg/mg tissue protein of the average of the normal control group.

In addition, it was confirmed that IL-1β of the single administration group of the test material, Neuromide (Examples 1 to 3) was 194.7 pg/mg tissue protein, 188.6 pg/mg tissue protein, and 151.5 pg/mg tissue protein on average, respectively, which were decreased as compared with the negative control group, and among them, Example 3 was statistically significantly decreased as compared with the negative control group.

It was confirmed that IL-1β of the single administration group of the comparative material, Donepezil (Comparative Example 3) and the combined administration group of the test material and the comparative material (Example 4) was 160.9 pg/mg tissue protein and 146.6 pg/mg tissue protein on average, respectively, which were statistically significantly decreased as compared with the negative control group.

5) Weight and General Symptom

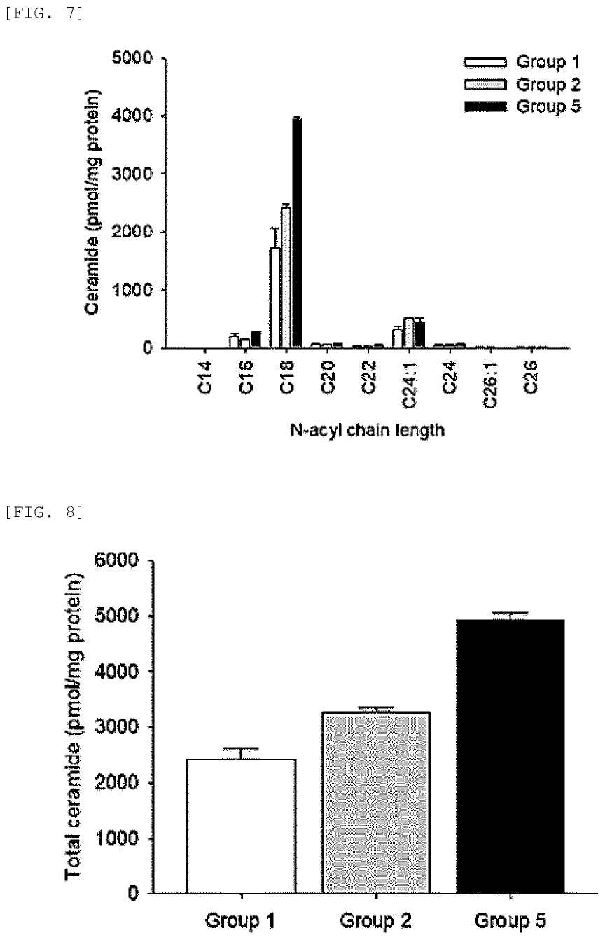

The results of measuring the weights of the objects of each experimental group twice a week during the test are shown in the following Tables 6 and 7 and FIG. 6:

TABLE 6
Results of measuring weight

| | Experimental | | | | | | |
|---|---|---|---|---|---|---|---|
| | group | Administration | Weight (g) | | | | |
| | Serial | dose | Time after administration (day) | | | | |
| | number | (mg/kg) | 0 | 1 | 5 | 8 | 12 |
| Comparative | G1 | — | 34.8 ± 0.5 | 34.9 ± 0.4 | 36.3 ± 0.6 | 36.5 ± 0.7 | 37.4 ± 0.9 |
| Example 1 | | | | | | | |
| Comparative | G2 | — | 34.9 ± 0.4 | 35.1 ± 0.7 | 36.3 ± 0.4 | 37 ± 0.6 | 37.4 ± 0.7 |
| Example 2 | | | | | | | |
| Example 1 | G3 | 16 | 34.9 ± 0.4 | 35.1 ± 0.4 | 35.9 ± 0.4 | 36.4 ± 0.5 | 36.4 ± 0.5 |
| Example 2 | G4 | 40 | 34.9 ± 0.4 | 35.5 ± 0.4 | 36 ± 0.3 | 36.7 ± 0.4 | 37.2 ± 0.4 |
| Example 3 | G5 | 100 | 34.9 ± 0.4 | 35.3 ± 0.5 | 36.2 ± 0.5 | 36.7 ± 0.7 | 37.6 ± 0.5 |
| Comparative | G6 | 1 | 34.9 ± 0.4 | 35.1 ± 0.4 | 35.9 ± 0.7 | 36.3 ± 0.8 | 36.7 ± 0.9 |
| Example 3 | | | | | | | |
| Example 4 | G7 | Mixture of | 35 ± 0.5 | 35.5 ± 0.4 | 36.3 ± 0.5 | 36.6 ± 0.6 | 37.3 ± 0.5 |
| | | Neuromide | | | | | |
| | | 100:Donepezil 1 | | | | | |

Data is expressed as a format of mean ± standard error.

TABLE 7
Results of measuring weight (continued from Table 1)

| | Experimental | | | | | | |
|---|---|---|---|---|---|---|---|
| | group | Administration | Weight (g) | | | | |
| | Serial | dose | Time after administration (day) | | | | |
| | number | (mg/kg) | 15 | 19 | 22 | 26 | 29 |
| Comparative | G1 | — | 37.5 ± 1 | 37.7 ± 1 | 38 ± 1 | 37.4 ± 1.2 | 37.1 ± 1.2 |
| Example 1 | | | | | | | |
| Comparative | G2 | — | 37.4 ± 0.8 | 37 ± 0.6 | 37.1 ± 0.6 | 37.3 ± 0.6 | 37.2 ± 0.6 |
| Example 2 | | | | | | | |
| Example 1 | G3 | 16 | 36.3 ± 0.6 | 37.3 ± 0.7 | 37.2 ± 1 | 37.2 ± 0.8 | 37.1 ± 0.9 |
| Example 2 | G4 | 40 | 37.5 ± 0.4 | 37.8 ± 0.4 | 38 ± 0.4 | 38.3 ± 0.4 | 37.9 ± 0.5 |
| Example 3 | G5 | 100 | 38 ± 0.6 | 38.6 ± 0.6 | 38.4 ± 0.7 | 38.8 ± 0.6 | 38.9 ± 0.8 |
| Comparative | G6 | 1 | 36.5 ± 1.1 | 37.2 ± 0.9 | 37.3 ± 0.9 | 37.8 ± 0.7 | 37.1 ± 0.7 |
| Example 3 | | | | | | | |
| Example 4 | G7 | Mixture of | 37.5 ± 0.6 | 37.4 ± 0.6 | 37.4 ± 0.6 | 37.5 ± 0.7 | 37.3 ± 0.9 |
| | | Neuromide | | | | | |
| | | 100:Donepezil 1 | | | | | |

Data is expressed as a format of mean ± standard error.

As confirmed therefrom, the weight was increased in all experimental group and no objects showing an abnormal symptom occurred during the test.

6) Analysis of Ceramide Content in Brain Tissue

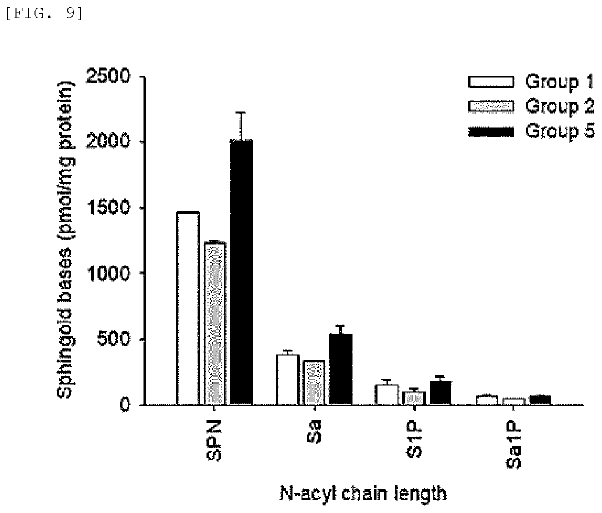

The results of analyzing the ceramide content in a brain tissue by chain length are shown in the following Table 8 and FIGS. 7 and 8:

TABLE 8
Ceramide content

| | Experimental | | | | | | | | | | | |
|---|---|---|---|---|---|---|---|---|---|---|---|---|
| | group | | | | | | | | | | | |
| | Serial | | N-acyl chain length | | | | | | | | | |
| | number | | C14 | C16 | C18 | C20 | C22 | C24:1 | C24 | C26:1 | C26 | Total |
| Comparative | G1 | Forebrain 1-2 | 3.80 | 247.12 | 1494.55 | 79.76 | 42.43 | 359.18 | 60.72 | 5.37 | 12.74 | 2305.68 |
| Example 1 | | Forebrain 1-3 | 1.99 | 175.83 | 1967.47 | 49.00 | 29.49 | 271.28 | 44.52 | 8.00 | 14.62 | 2562.21 |
| | | Average | 2.90 | 211.48 | 1731.01 | 64.38 | 35.96 | 315.23 | 52.62 | 6.69 | 13.68 | 2433.94 |
| | | S.D. | 1.28 | 50.41 | 334.40 | 21.75 | 9.15 | 62.15 | 11.45 | 1.86 | 1.33 | 181.39 |
| Comparative | G2 | Forebrain 2-2 | 2.30 | 154.64 | 2464.30 | 60.57 | 37.30 | 524.28 | 64.21 | 6.17 | 10.01 | 3323.78 |
| Example 2 | | Forebrain 2-3 | 1.79 | 150.56 | 2398.69 | 61.18 | 31.36 | 498.30 | 43.56 | 4.94 | 5.43 | 3195.82 |
| | | Average | 2.04 | 152.60 | 2431.50 | 60.88 | 34.33 | 511.29 | 53.89 | 5.55 | 7.72 | 3259.80 |
| | | S.D. | 0.36 | 2.88 | 46.39 | 0.43 | 4.20 | 18.37 | 14.60 | 0.87 | 3.24 | 90.48 |
| Example 3 | G5 | Forebrain 5-2 | 2.92 | 288.68 | 3980.71 | 94.95 | 60.05 | 498.48 | 77.31 | 4.78 | 12.46 | 5020.32 |
| | | Forebrain 5-3 | 2.50 | 279.51 | 3932.52 | 90.81 | 43.51 | 408.35 | 59.29 | 4.40 | 9.72 | 4830.60 |
| | | Average | 2.71 | 284.09 | 3956.61 | 92.88 | 51.78 | 453.42 | 68.30 | 4.59 | 11.09 | 4925.46 |
| | | S.D. | 0.29 | 6.48 | 34.07 | 2.93 | 11.70 | 63.73 | 12.74 | 0.27 | 1.94 | 134.16 |

As confirmed therefrom, the ceramide synthesis amount was significantly increased in the experimental group to which 100 mg/kg of Neuromide was administered, and in particular, C18 ceramide was the majority as compared with ceramide of other chain lengths.

7) Analysis of Sphingoid Base Content in Brain Tissue

The results of analyzing the sphingoid base content in a brain tissue are shown in the following Table 9 and FIG. 9:

TABLE 9
Sphingoid base content

| | Experimental | | | | | |
|---|---|---|---|---|---|---|
| | group | | Sphingoid base | | | |
| | Serial | | Sphingosine | Sphinganine | | |
| | number | | (SPN) | (Sa) | S1P | Sa1P |
| Comparative | G1 | Forebrain 1-2 | 1451.44 | 361.16 | 179.59 | 61.71 |
| Example 1 | | Forebrain 1-3 | 1472.21 | 402.83 | 127.14 | 68.50 |
| | | Average | 1461.82 | 382.00 | 153.36 | 65.10 |
| | | S.D. | 14.69 | 29.47 | 37.09 | 4.80 |
| Comparative | G2 | Forebrain 2-2 | 1226.16 | 337.69 | 114.78 | 50.64 |
| Example 2 | | Forebrain 2-3 | 1248.77 | 340.52 | 76.75 | 44.09 |
| | | Average | 1237.96 | 339.10 | 95.76 | 47.37 |
| | | S. D. | 15.28 | 2.00 | 26.89 | 4.63 |
| Example 3 | G5 | Forebrain 5-2 | 1861.23 | 506.47 | 159.43 | 69.16 |
| | | Forebrain 5-3 | 2158.78 | 585.01 | 208.05 | 69.98 |
| | | Average | 2010.00 | 545.74 | 183.74 | 69.57 |
| | | S.D. | 210.40 | 55.53 | 34.38 | 0.58 |

As confirmed therefrom, the synthesis amount of sphingosine in the experimental group of G1 to which 100 mg/kg of Neuromide was administered was significantly increased from 1461.82 to 2010.00. In particular, it was confirmed that S1P in the state of being greatly decreased in the neurodegenerative brain disease was greatly decreased from 153.36 of the normal control group to 95.76 of the negative control group, and when 100 mg/kg of Neuromide was administered, it was 183.74, which was increased by 20% or more as compared with the normal control group.

From the above results, as results of administering scopolamine which temporarily reduces memory and learning ability and then repeatedly administering the test material according to the present invention to measure the effect on memory improvement by the passive avoidance test, according to the present invention, it was confirmed that the average time taken to move to the room where there was electric shock was statistically significantly increased in all of the test material administration group, the comparative material administration group, and the combined administration group of the test material and the comparative material in all doses.

In conclusion, it was considered that in the cognitive ability and memory impairment model, the activity of acetylcholinesterase is inhibited in a brain tissue by repeatedly administering Neuromide as the test material, thereby increasing the amount of acetylcholine and decreasing the amount of inflammatory cytokine to protect brain cells, and thus, an effect of improving cognitive ability and memory was shown.

Hereinabove, although the present invention has been described by specified matters and specific exemplary embodiments, they have been provided only for assisting in the entire understanding of the present invention. Therefore, the present invention is not defined by the specific matters limited to the exemplary embodiments. Various modifications and changes may be made by those skilled in the art to which the present invention pertains from this description.

Therefore, the spirit of the present invention should not be limited to the above-described exemplary embodiments, and the following claims as well as all modified equally or equivalently to the claims are intended to fall within the scope and spirit of the invention.

1. A pharmaceutical composition for improving cognitive ability or enhancing memory comprising N-palmitoyl serinol as an effective component.

2. The pharmaceutical composition for improving cognitive ability or enhancing memory of claim 1, wherein the pharmaceutical composition is for improving or treating neurodegenerative brain diseases or neuroinflammatory diseases.

3. The pharmaceutical composition for improving cognitive ability or enhancing memory of claim 2, wherein the disease is selected from the group consisting of autism, Alzheimer's disease, Parkinson's disease, peripheral neuropathy, amyotrophic lateral sclerosis, ischaemia, stroke, and multiple sclerosis.

4. The pharmaceutical composition for improving cognitive ability or enhancing memory of claim 1, wherein the pharmaceutical composition is a formulation of a preparation of oral administration or a preparation of parenteral administration.

5. The pharmaceutical composition for improving cognitive ability or enhancing memory of claim 4, wherein the formulation of the preparation of oral administration is tablets, pills, hard/soft capsules, liquid, suspension, emulsifiers, syrup, granules, or elixirs.

6. The pharmaceutical composition for improving cognitive ability or enhancing memory of claim 4, wherein the formulation of the preparation of parenteral administration is a sterile aqueous or oily solvent for intravenous, subcutaneous, sublingual, or intramuscular administration.

7. A health functional food for improving cognitive ability or enhancing memory comprising the pharmaceutical composition of claim 1.

8. The health functional food for improving cognitive ability or enhancing memory of claim 7, wherein a formulation of the health functional food is powder, granules, tablets, capsules, or beverages.

9. A composite pharmaceutical composition for improving cognitive ability or enhancing memory comprising N-palmitoyl serinol and an active material for improving cognitive ability or enhancing memory.

10. The composite pharmaceutical composition for improving cognitive ability or enhancing memory of claim 9, wherein the composite pharmaceutical composition includes:
first particles including N-palmitoyl serinol; and
second particles including the active material for improving cognitive ability or enhancing memory.

11. The composite pharmaceutical composition for improving cognitive ability or enhancing memory of claim 10, wherein the composite pharmaceutical composition further includes a sterol-based compound, a higher fatty acid, and a higher fatty acid alcohol.

12. The composite pharmaceutical composition for improving cognitive ability or enhancing memory of claim 11, wherein 10 to 1000 parts by weight of the sterol-based compound, 100 to 3000 parts by weight of the higher fatty acid, 100 to 5000 parts by weight of the higher fatty acid alcohol, and 1 to 500 parts by weight of the active material are included by mixing with respect to 100 parts by weight of N-palmitoyl serinol.

13. The composite pharmaceutical composition for improving cognitive ability or enhancing memory of claim 10, wherein the composite pharmaceutical composition forms a stable crystal lamella micro-encapsulation system (MES) or orthorhombic crystalline phase.

14. The composite pharmaceutical composition for improving cognitive ability or enhancing memory of claim 9, wherein the active material is any one or more selected from the group consisting of Donepezil, Rivastigmine, and Galantamine.

15. A method of preparing a composite pharmaceutical composition for improving cognitive ability or enhancing memory, the method comprising:
preparing first particles including N-palmitoyl serinol; and
preparing second particles including an active material for improving cognitive ability or enhancing memory.

16. The method of preparing a composite pharmaceutical composition for improving cognitive ability or enhancing memory of claim 15, wherein the active material is included in a form of the active material or a pharmaceutically acceptable salt thereof,
wherein the first particles and the second particles are mixed at a weight ratio of 100:0.5 to 1.5, and
wherein the first particles and the second particles release N-palmitoyl serinol and the active material continuously for 12 hours to 24 hours to maintain an effect of improving diseases related to cognitive impairment or memory loss.

17. (canceled)

18. (canceled)

19. The method of preparing a composite pharmaceutical composition for improving cognitive ability or enhancing memory of claim 15, wherein the disease related to cognitive impairment or memory loss is selected from the group consisting of autism, Alzheimer's disease, Parkinson's disease, peripheral neuropathy, amyotrophic lateral sclerosis, ischaemia, stroke, and multiple sclerosis.

20. The method of preparing a composite pharmaceutical composition for improving cognitive ability or enhancing memory of claim 15, wherein the first particles and the second particles are mixed in a uniform form to form a stable crystal lamella micro-encapsulation system (MES) or orthorhombic crystalline phase.

21. An administration method for improving cognitive ability or memory decline, the method comprising: administrating the pharmaceutical composition of claim 1 to a subject.

22. The administration method for improving cognitive ability or memory decline of claim 21, wherein a dose of the pharmaceutical composition to be administered to the subject is 1 to 200 mg/kg based on the weight of the subject,
wherein an administration interval of the pharmaceutical composition to be administered to the subject is 12 hours or more and 240 hours or less, and
wherein the administration method includes oral administration, subcutaneous injection, and subcutaneous application.

23. (canceled)

24. (canceled)
